(19) United States
(12) Patent Application Publication
(10) Pub. No.: US 2014/0027466 A1
(43) Pub. Date: Jan. 30, 2014

(54) TEST STRIP ANALYZING APPARATUS HAVING WHEEL EJECTOR

(71) Applicants: In Hwan Choi, Suwon-si (KR); Jeong In Bag, Anyang Si (KR)

(72) Inventors: In Hwan Choi, Suwon-si (KR); Jeong In Bag, Anyang Si (KR)

(73) Assignee: PHILOSYS CO., LTD., Gunsan-si, Jeollabuk-do (KR)

(21) Appl. No.: 14/111,251

(22) PCT Filed: Apr. 28, 2011
(86) PCT No.: PCT/KR11/03160

§ 371 Date: Oct. 11, 2013

(30) Foreign Application Priority Data
Apr. 13, 2011 (KR) 10-2011-0034102

(51) Int. Cl. A61B 5/157; A61B 5/145

(52) U.S. Cl. CPC A61B 5/157; A61B 5/145; G01N 2035/00089
USPC 221/259

(57) ABSTRACT

Disclosed herein is a test strip analyzing apparatus having a wheel ejector. The test strip analyzing apparatus having a wheel ejector includes a body, a measurement unit, and an ejector. The measurement unit is disposed on one side of the body. The ejector is disposed in the measurement unit, and selectively inserts and discharges a test strip. Furthermore, the ejector includes a wheel that is rotatably mounted in the measurement unit so that a part of the wheel is exposed outside a top portion of the measurement unit and that selectively inserts and discharges the test strip using frictional force.

### TECHNICAL FIELD

The present invention relates, in general, to a test strip analyzing apparatus having a wheel ejector, and, more particularly, to a test strip analyzing apparatus having a wheel ejector, which can facilitate the insertion and discharging of a test strip and can prevent the apparatus and a user from being contaminated by residual bio-samples.

### BACKGROUND ART

A test strip analyzing apparatus is a device that allows a bio-sample, such as blood, to be applied to a test strip and analyzes information, such as blood glucose, pregnancy, viral infections and immunity. Such test strip analyzing apparatuses can be conveniently used by users, and therefore they are widely used for blood glucose meters and pregnancy testers.

Such test strip analyzing apparatuses are generally classified into test strip analyzing apparatuses using a photometric measuring method and test strip analyzing apparatuses using an electrochemical measuring method. The test strip analyzing apparatuses using a photometric measuring method require a long measuring time, allow the price to be low and produce a low added value per manufacturing cost, compared to the test strip analyzing apparatuses using an electrochemical measuring method, and therefore the test strip analyzing apparatuses using an electrochemical measuring method have been chiefly used recently.

When a electrochemical test strip analyzing apparatus is used, a test strip is loaded into the test strip analyzing apparatus, a bio-sample of an examinee is applied to the exposed side of the test strip, the applied bio-sample is tested using a test unit in the test strip analyzing apparatus, test results are displayed on a display outside the test strip analyzing apparatus, the test strip that was used is removed from the test strip analyzing apparatus, a new strip is loaded into the test strip analyzing apparatus, the test strip analyzing apparatus enters a standby state, and this process is repeated.

Here, since the test strip is removed from the test strip analyzing apparatus while a user directly grips the test strip, a bio-sample remaining in the test strip may stick to an examiner or an examinee him or herself, so that the bio-sample sticking to the hand contaminates a new strip while the new strip is loaded into the test strip analyzing apparatus, with the result that there is the problem of making a misdiagnosis. Furthermore, when excessive force is used while the test strip is being removed from the test strip analyzing apparatus, there occurs the problem of the test strip tearing. Additionally, there arises the problem of separating the test strip analyzing apparatus in order to remove the damaged strip from the test strip analyzing apparatus.

Accordingly, recently, there has been proposed a test strip analyzing apparatus that is provided with an ejector so that the test strip that was used can be removed from the test strip analyzing apparatus without requiring a user to directly grip the strip, thereby facilitating the discharging of the test strip and enabling sanitary tests to be conducted.

The ejector of the above conventional test strip analyzing apparatus is configured to push a strip insertion part, through which a test strip is inserted, outwards. A spring is compressed when the strip insertion part is pushed outwards, and restores the insertion part to its original state when the spring is restored after being compressed.

However, when the strip insertion part is pushed, the pushing of the strip insertion part is inconvenient, the speed with which a test strip is discharged is very low, and the operation of the ejector is difficult because the structure of the ejector is very complicated.

### DISCLOSURE OF INVENTION

### Technical Problem

Accordingly, the present invention has been made keeping in mind the above problems occurring in the prior art, and an object of the present invention is to provide a test strip analyzing apparatus having a wheel ejector, which can facilitate the insertion and discharging of a test strip and can prevent the apparatus and a user from being contaminated by residual bio-samples.

### Solution to Problem

In order to accomplish the above object, the present invention provides a test strip analyzing apparatus having a wheel ejector, including a body, a measurement unit disposed on one side of the body, and an ejector disposed in the measurement unit and configured to selectively insert and discharge a test strip, wherein the ejector includes a wheel that is rotatably mounted in the measurement unit so that a part of the wheel is exposed outside a top portion of the measurement unit and that selectively inserts and discharges the test strip using frictional force.

The wheel may be made of a silicon, rubber or synthetic rubber material that is capable of producing a large amount of frictional force.

A rugged portion may be formed on a surface of the wheel so as to prevent slipping.

A cover may be provided on the top portion of the measurement unit so as to cover the exposed portion of the wheel.

### Advantageous Effects of Invention

According to the test strip analyzing apparatus having a wheel ejector according to the present invention, the test strip is inserted and discharged by pressing and then rotating the wheel using a user's finger, so that the strip is conveniently inserted, the speed with which the strip is discharged is very fast, and the strip can be accurately and easily discharged by the single action of rotating the wheel.

Furthermore, the structure of the ejector is simple and the manipulation of the ejector is easy, so that the use of the ejector is very convenient. An examiner does not need to directly grip the strip that was used, so that sanitary tests can be conducted. Moreover, the apparatus and a user can be prevented from being contaminated by residual bio-samples.

### BRIEF DESCRIPTION OF DRAWINGS

The above and other objects, features and advantages of the present invention will be more clearly understood from the following detailed description taken in conjunction with the accompanying drawings, in which:

FIG. 1 is a perspective view showing a test strip analyzing apparatus having a wheel ejector according to the present invention;

FIG. 2 is a sectional view showing the insertion of a test strip into the test strip analyzing apparatus having a wheel ejector according to the present invention by the operation of a wheel;

FIG. 3 is a sectional view showing the discharging of a test strip from the test strip analyzing apparatus having a wheel ejector according to the present invention by the operation of the wheel; and

FIG. 4 is a partial enlarged view showing a cover disposed over the exposed part of the wheel in the test strip analyzing apparatus having a wheel ejector according to the present invention.

### MODE FOR THE INVENTION

Reference now should be made to the drawings, throughout which the same reference numerals are used to designate the same or similar components.

A test strip analyzing apparatus having a wheel ejector according to the present invention will be described in detail below with reference to the accompanying drawings.

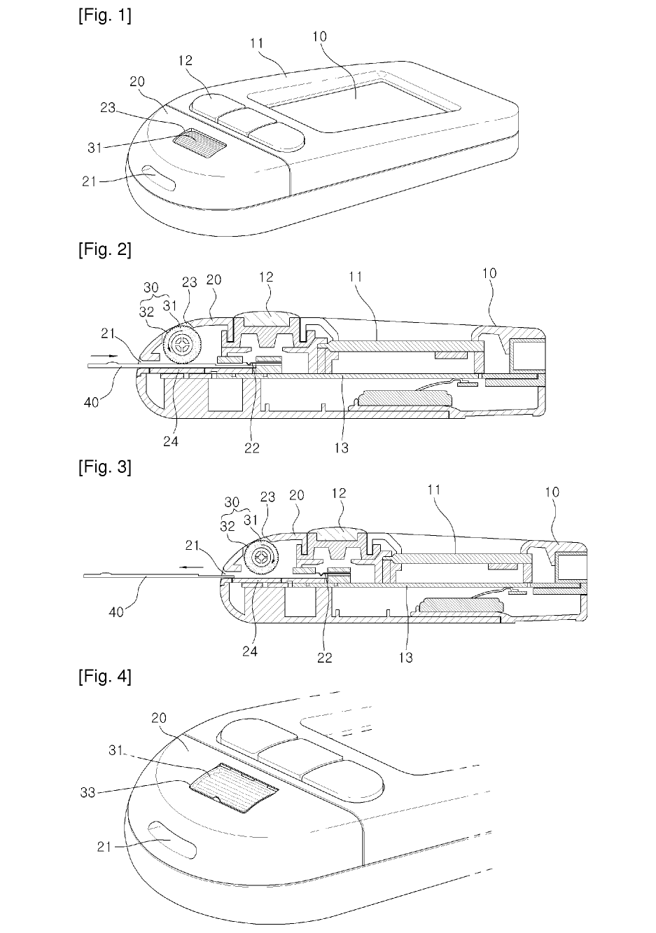

FIG. 1 is a perspective view showing a test strip analyzing apparatus having a wheel ejector according to the present invention. FIG. 2 is a sectional view showing the insertion of a test strip into the test strip analyzing apparatus having a wheel ejector according to the present invention by the operation of a wheel. FIG. 3 is a sectional view showing the discharging of a test strip from the test strip analyzing apparatus having a wheel ejector according to the present invention by the operation of the wheel. FIG. 4 is a partial enlarged view showing a cover disposed over the exposed part of the wheel in the test strip analyzing apparatus having a wheel ejector according to the present invention.

As shown in FIGS. 1 to 3, a test strip analyzing apparatus having a wheel ejector according to the present invention includes a body 10, a measurement unit 20 disposed on one side of the body 10, and an ejector 30 disposed in the measurement unit 20 and configured to selectively receive and discharge a strip.

Here, the body 10 includes a display unit 11 disposed on the top portion of the body 10 and configured to display measured biometric values or a variety of types of information related to biometric information such as a variety of types of image information based on measured values, a graph representative of the variations of measured values and the sequence of the measurement of biometric information, and a manipulation unit 12 disposed at an appropriate location on one side of the display unit 11 and provided with a power key and function keys.

The measurement unit 20 is provided on one side of the body 10.

The measurement unit 20 includes a strip reception slit 21 formed at one end of the measurement unit 20 and configured to receive a test strip and a contract terminal 22 disposed inside the measurement unit 20 and configured to come into contact with one end of the test strip 40 when the test strip 40 is inserted into the strip reception slit 21. The contract terminal 22 is connected to the circuit board 13 of the display unit 11.

Furthermore, a through hole 23 is formed in the top portion of the measurement unit 20. The ejector 30 is fitted into the through hole 23 and functions to selectively receive and discharge the test strip 40 which is inserted into the strip reception slit 21.

The ejector 30 includes a wheel 31 that is rotatably disposed inside the measurement unit 20 so that a part of the wheel 31 is exposed outside the top portion of the measurement unit 20 via the through hole 23 and the remaining part thereof is accommodated in the measurement unit 20.

Here, the wheel 31 is disposed to be spaced apart from the inside bottom surface 24 of the measurement unit 20 by a predetermined distance. It is preferable to determine the distance so that the test strip 40 is tightly inserted between the wheel 31 and the bottom surface 24.

Furthermore, the wheel 31 is preferably made of a silicon, rubber or synthetic rubber material capable of producing a large amount of frictional force in order to enable the test strip 40 to be selectively inserted and discharged.

Furthermore, a rugged portion 32, such as a knurled portion or a portion with protrusions, is preferably formed on the surface of the wheel 31 in order to prevent the user's finger from slipping when the user presses and rotates the wheel 31 using his or her finger and make the wheel 31 be controlled smoothly.

Furthermore, a cover 33 is preferably provided over the measurement unit 20 in order to cover the exposed part of the wheel 31 (see FIG. 4).

The cover 33 functions to prevent impurities from entering into the measurement unit 20 via the through hole 23, prevent contamination from being produced by residual bio-samples and protect the wheel 31.

Here, the cover 33 may be configured such that one side thereof is hinged to the top portion of the measurement unit 20 and the cover 33 can be rotated around a hinge shaft, as shown in FIG. 4. Alternatively, although not shown in the drawings, the cover 23 may be configured such that a guide is provided across the top portion of the measurement unit 20 and the cover 23 is engaged with and then slides through the guide.

The use of the test strip analyzing apparatus having a wheel ejector, which is configured as described above, will now be described.

First, as shown in FIG. 2, when a test strip 40 is inserted into the strip reception slit 21 of the test strip analyzing apparatus, the strip 40 is located beneath the wheel 31. Thereafter, when the user presses the wheel 31, a part of which is exposed outside the measurement unit 20, and rotates it in one direction (in the direction of an arrow), the wheel 31 is rotated and moves the strip 40 located beneath the wheel 31 inwards using the frictional force therebetween so that one end of the strip 40 is brought into contact with the contract terminal 22 provided inside the measurement unit 20.

When one side of the strip 40 is engaged with the measurement unit 20, a bio-sample of an examinee is applied to the remaining side of the strip 40 which is exposed outside the measurement unit 20.

The bio-sample applied to the strip 40 is measured using the measurement unit 20, measured values are displayed on the display unit 11, the examinee checks the measured values on the display unit 11, and then a test is terminated.

After the test has been terminated, the user presses the wheel 31 exposed outside the top portion of the measurement unit 20 and rotates it in the other direction (in the direction of the arrow), as shown in FIG. 3. The rotated wheel 31 moves the strip 40 located beneath the wheel 31 in a direction toward the strip reception slit 21 using frictional force, and therefore discharges the strip 40 from the measurement unit 20.

As described above, the present invention is configured such that the strip 40 is inserted and discharged by pressing and then rotating the wheel 31 using the user's finger, so that the strip 40 is conveniently inserted, the speed with which the strip 40 is discharged is very fast, and the strip 40 can be accurately discharged by the single action of rotating the wheel 31.

Furthermore, since the structure of the ejector 30 is simple and the manipulation of the ejector 30 is easy, the use of the ejector 30 is very convenient. Moreover, since an examiner does not need to directly grip the strip 40 used, sanitary tests can be conducted.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A test strip analyzing apparatus having a wheel ejector, comprising a body, a measurement unit disposed on one side of the body, and an ejector disposed in the measurement unit and configured to selectively insert and discharge a test strip,
wherein the ejector comprises a wheel that is rotatably mounted in the measurement unit so that a part of the wheel is exposed outside a top portion of the measurement unit and that selectively inserts and discharges the test strip using frictional force.

2. The test strip analyzing apparatus as set forth in claim 1, wherein the wheel is made of a silicon, rubber or synthetic rubber material that is capable of producing a large amount of frictional force.

3. The test strip analyzing apparatus as set forth in claim 1, wherein a rugged portion is formed on a surface of the wheel so as to prevent slipping.

4. The test strip analyzing apparatus as set forth in claim 1, wherein a cover is provided on a top portion of the measurement unit so as to cover the exposed portion of the wheel.
